(19) United States
(12) Patent Application Publication
(10) Pub. No.: US 2017/0281382 A1
(43) Pub. Date: Oct. 5, 2017

(54) DELIVERY SYSTEMS WITH INTRODUCER AND DISTAL SHEATHS AND METHODS OF USE

(71) Applicant: Bolton Medical, Inc., Sunrise, FL (US)

(72) Inventors: Timothy Lostetter, Sunrise, FL (US); Scott Lyle Rush, Sunrise, FL (US)

(21) Appl. No.: 15/478,424

(22) Filed: Apr. 4, 2017

Related U.S. Application Data
(60) Provisional application No. 62/318,427, filed on Apr. 5, 2016.

(51) Int. Cl. A61F 2/966; A61M 39/06; A61F 2/07

(52) U.S. Cl. CPC A61F 2/966; A61F 2/07; A61M 39/06; A61F 2002/9517

(57) ABSTRACT

A delivery system and method of its use delivers a stent graft prosthesis to a surgical site, such as an abdominal, thoracic or thoraco-abdominal aortic aneurysm. The delivery system includes a handle defining a conduit, an introducer sheath extending distally through the handle, a substantially straight inner control tube extending through the conduit of the handle and from the distal end of the introducer sheath, and a distal sheath that extends through and beyond a distal end of the introducer sheath. The distal sheath can have, in one embodiment, a diameter equal to or less than that of the introducer sheath. Retraction of the distal sheath through the introducer sheath and the handle exposes a stent graft prosthesis extending about the inner control tube. Removal of the inner control tube from the introducer sheath allows for implantation of additional components through the introducer sheath.

### RELATED APPLICATION

This application claims the benefit of U.S. Provisional Application No. 62/318,427, filed on Apr. 5, 2016. The entire teachings of the above application are incorporated herein by reference.

### BACKGROUND OF THE INVENTION

Stent graft delivery systems are designed to treat abdominal aortic aneurysms (AAA), thoracic aortic aneurysms (TAA) and thoraco-abdominal aortic aneurysms (TAAA). Successful placement of components of a prosthesis is critical to bypass the aneurysm. Prostheses can be deployed as multiple units, each requiring separate deployment from a delivery system. Prevention of extracorporeal blood flow while changing delivery devices during implantation of a main prosthesis, or in the absence of blood vessel filling catheters or accessories, can be accomplished by hemostasis valves.

Thus, there is a need to develop new, useful and effective delivery systems, components and methods having hemostasis valves to treat AAA.

### SUMMARY OF THE INVENTION

The present invention relates to a delivery system, and methods of using the delivery system to treat vascular damage, in particular AAA, TAA and TAAA.

In one embodiment, the invention is a delivery assembly that includes a handle defining a conduit, an introducer sheath extending distally from handle, the introducer sheath having a proximal end at the handle, and a distal end that is distal to the handle. A substantially straight inner control tube extends through the conduit of the handle and the introducer sheath, and has a proximal end that is proximal to the handle at a distal end that is distal to the distal end of the introducer sheath, a tip fixed to the distal end of inner control tube, and a distal sheath that extends through the handle and the introducer sheath, and distally from the distal end of introducer sheath and about the inner control tube to the tip, wherein the distal sheath has a diameter about equal to or less than that of the introducer sheath, wherein retraction of the distal sheath through the introducer sheath and the handle exposes the inner control tube. In one embodiment, the distal sheath has a diameter larger than introducer sheath, but still allows for retraction through the introducer sheath.

In another embodiment, the invention is a method for delivering a stent graft to a surgical site. The method includes directing a distal end of a delivery assembly of the invention to an abdominal aortic aneurysm or a thoracic aortic aneurysm, and retracting the distal sheath from a stent graft restrained by the delivery assembly, thereby exposing the stent graft to the aneurysm.

This invention has many advantages. For example, a distal sheath that extends beyond the introducer sheath and having a diameter that is equal to or less than, or in some cases larger than, that of an introducer sheath need not be advanced from the introducer sheath to a surgical site, but, rather, can be advanced with advancement of the remainder of the delivery assembly in a patient until the stent graft prosthesis is at the surgical site for implantation. Once the stent graft is deployed, additional components of the delivery device, such as the inner control tube, can be removed from the patient through the introducer sheath, leaving the introducer sheath behind and available for other purposes, such as for implanting additional components of a fully assembled prosthesis, of which the stent graft is one part. Thus, the delivery assembly of the invention can be employed to treat abdominal aortic aneurysms (AAA), thoracic aortic aneurysms (TAA) and thoraco-abdominal aortic aneurysm (TAAA), to thereby avoid complications and death consequent to life-threatening vascular conditions.

### BRIEF DESCRIPTION OF THE DRAWINGS

The foregoing will be apparent from the following more particular description of example embodiments of the invention, as illustrated in the accompanying drawings, in which like reference characters refer to the same parts throughout the different views. The drawings are not necessarily to scale, emphasis instead being placed upon illustrating embodiments of the present invention.

FIG. 1 is a perspective view of one embodiment of a delivery assembly of the invention.

FIG. 1A is a detail of the distal end of an introducer sheath of the delivery assembly of FIG. 1 after retraction from an inner control tube of that embodiment.

FIG. 2 is a partial cross-sectional view of one embodiment of the delivery assembly of FIG. 1 prior to deployment of a stent graft prosthesis from within a distal sheath of the delivery assembly.

FIGS. 2A-2C are detail views of the embodiment represented in FIG. 2.

FIG. 3 is a cross-sectional view, in perspective, of a hemostasis valve suitable for use with the invention.

FIG. 3A is a detail of FIG. 3 showing the relationship between the hemostasis valve, the distal sheath, an intermediate tube, an inner control tube, and an apex release lumen of one embodiment of the invention.

FIG. 4 is a cross-sectional view of the delivery assembly of the invention of FIG. 2, wherein the distal sheath has been partially retracted from the tip of the delivery assembly through the introducer sheath and handle of the assembly.

FIG. 5 is a cross-sectional view of the delivery assembly following further retraction of the distal sheath and opening of a stent capture device at the tip.

FIG. 6 is a perspective view of the delivery assembly of FIG. 1, wherein the distal sheath has been fully retracted from the stent graft.

FIG. 7 is a cross-sectional view of the delivery assembly of FIGS. 5 and 6 after complete retraction of the distal sheath through the introducer sheath and the handle.

FIGS. 8A and 8B are perspective views of the delivery assembly of FIGS. 1-7, wherein the screw is removed from the handle and hemostasis valve, to permit retraction of the handle, the distal sheath, the intermediate tube, the apex release lumen and the inner control tube from the surgical site of the patient.

FIG. 9 is a cross-sectional view of the delivery assembly as shown in FIG. 7 after partial retraction of the inner control tube, the capture device (when open), and the tip.

FIGS. 10A and 10B are perspective views of the distal assembly of FIG. 6 after complete retraction of the handle of the distal sheath from the hemostasis valve of the introducer sheath.

### DETAILED DESCRIPTION OF THE INVENTION

The invention is generally directed to delivery systems for use in implantation of a stent graft at a site of a AAA, TAA or TTAA.

The features and other details of the invention, either as steps of the invention or as combinations of parts of the invention will now be more particularly described and pointed out in the claims. It will be understood that the particular embodiments of the invention are shown by way of illustration and not as limitations of the invention. The principle features of this invention can be employed in various embodiments without departing from the scope of the invention.

“Proximal” means, when reference is made to a delivery system or a component of a delivery system, or relatively close to the clinician using the device. Conversely, “distal” means, when reference is made to a delivery system or a component of a delivery system, relatively far from the clinician using the device.

When reference is made to a “stent” or a “stent graft system,” “proximal” means that the end of the stent or stent graft system that is relatively close to the heart of the patient, and “distal” means that end of the stent or stent graft system that is relatively far from the heart of the patient.

For clarity, the word “proximate” means close to, as opposed to the meanings ascribed to “proximal” or “distal” above with respect to either the vascular repair device or delivery system.

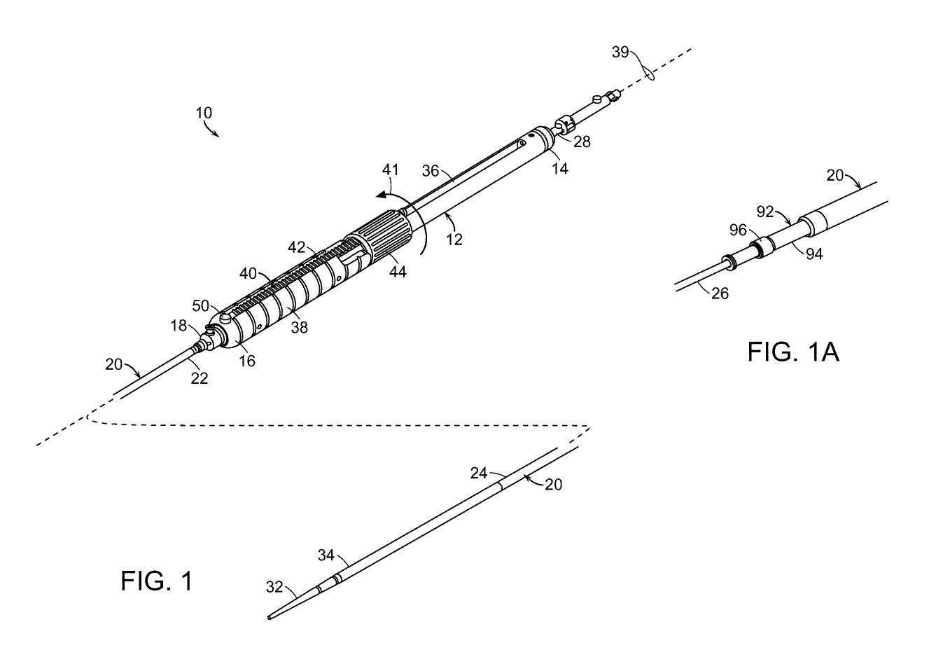

In one embodiment, represented in FIG. 1, delivery assembly 10 of the invention includes handle 12. Handle 12 has proximal end 14 and distal end 16, and defines a conduit. Handle 12 is formed of a suitable material, such as anodized aluminum, polystyrene, polypropylene, polyethylene, polyvinyl chloride, nylon polycarbonate, polyester, polyurethane, or any other suitable engineering plastic, as is known to those skilled in the art. Hemostasis valve component 18 is releasably fixed to distal end 16 of handle 12 by release pin 50. One example of a suitable hemostasis valve is described in U.S. Pat. No. 9,439,751, issued on Sep. 13, 2016, the relevant teachings of which are incorporated herein by reference in their entirety.

Introducer sheath 20 extends distally from hemostasis valve 18, to which it is fixed. Introducer sheath 20 includes proximal end 22 at hemostasis valve 18 and distal end 24 that is distal to handle 12. Introducer sheath 20 is fabricated of suitable material, preferably flexible material such as DACRON®, polyethylene terephthalate (PET, also referred to as PETE), fluorinated ethylene propylene (FEP), or polytetrafluoroethylene (TEFLON®), as is known to those of skill in the art. In one particular embodiment, introducer sheath 20 is fabricated as a tube formed of coiled Nitinol, a high density polyether (HDPE)/polyether block amide (e.g., PEBAX®), a stainless steel (SS) coil and aramid fibers. Introducer sheath 20 defines a conduit. The conduits of handle 12 and introducer sheath 20 are essentially coaxial.

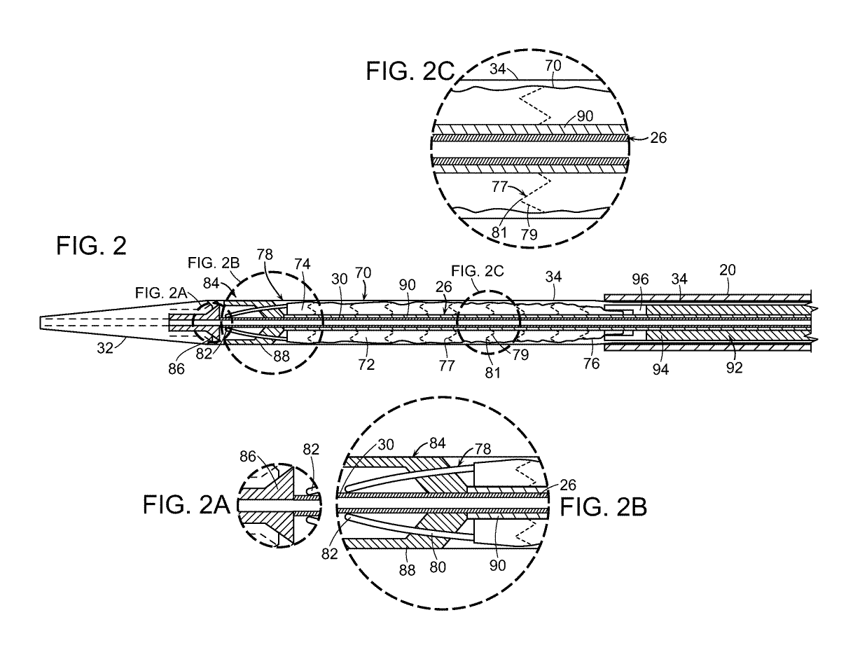

Referring to FIGS. 1A and 2, inner control tube 26 extends through the conduits of handle 12 and introducer sheath 20. Inner control tube 26 has proximal end 28 that is proximal to handle 12 and distal end 30 that is distal to distal end 24 of introducer sheath 20. Inner control tube 26 defines a luminal conduit.

Tip 32 is fixed to distal end 30 of inner control tube 26 (FIG. 2A) and defines a conduit through which a guidewire (not shown) can be directed. Tip 32 is formed of a suitable material, such as TECOTHANE® aromatic polyether-based thermoplastic polyurethane (TPU), as is known to those skilled in the art.

Distal sheath 34 extends through handle 12 and introducer sheath 20, and also extends distally from distal end 24 of introducer sheath 20 and about inner control tube 26 to tip 32. Distal sheath 34 has a diameter equal to or less than that of introducer sheath 20, whereby retraction of distal sheath 34 through introducer sheath 20 and handle 12 exposes inner control tube 26. Distal sheath 34 is formed of a suitable material, such as a suitable flexible material of the same or similar material as that of introducer sheath 20, or as is known to those skilled in the art. In one embodiment, distal sheath 34 is fabricated of a material that is more flexible than that of introducer sheath 20.

Inner control tube 26 can be retracted independently through introducer sheath 20 and handle 12, either manually or by a suitable mechanism, such as is described in U.S. Pat. No. 8,998,970, the relevant teachings of which are incorporated herein by reference in their entirety.

Typically, tip 32 has a smaller outside diameter than the conduit defined by introducer sheath 20 and the conduit defined by handle 12, so that distal sheath 34, inner control tube 26 and tip 34 can be retracted through introducer sheath 20 and the conduit defined by hemostasis valve 18, and thereby be removed from within introducer sheath 20 and hemostasis valve 18.

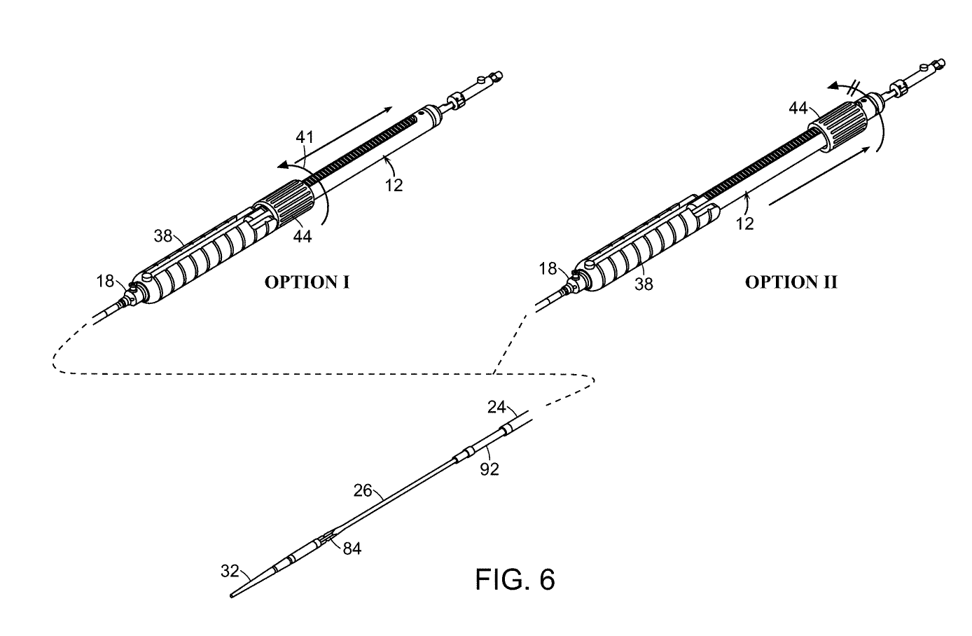

Handle 12 also defines track 36 along distal grip 38 of handle 12, as shown in FIG. 1. Internal lead screw assembly 40 is located within the conduit of handle 12 and is moveable along major axis 39 of handle 12. Internal lead screw assembly 40 further includes threaded portion 42 that extends through track 36. Lead screw nut 44 extends about handle 12 and is threadably engaged with threaded portion 42 of internal lead screw assembly 40, whereby rotation of lead screw nut 44 while abutting distal grip 38, as shown by arrow 41, causes movement of the internal lead screw assembly 40 relative to handle 12, as shown by Option I of FIG. 6. Lead screw nut 44 also is slidable along handle 12 while engaged with internal lead screw assembly 40, as shown by Option II in FIG. 6, without the need to rotate lead screw nut 44, thereby providing at least two independent mechanisms for causing movement of internal lead screw assembly 40 relative to handle 12. Internal lead screw assembly 40 of the stent graft delivery system 10 of the invention defines an opening essentially coaxial with handle 12, wherein inner control tube 26 extends through internal lead screw assembly 40.

Inner control tube 26 is fixed to handle 12. Distal sheath 34 extends about inner control tube 26 and is fixed, either directly or indirectly, to internal lead screw assembly 40, whereby movement of internal lead screw assembly 40 relative to the handle 12 causes relative longitudinal movement of distal sheath 34 relative to introducer sheath 20.

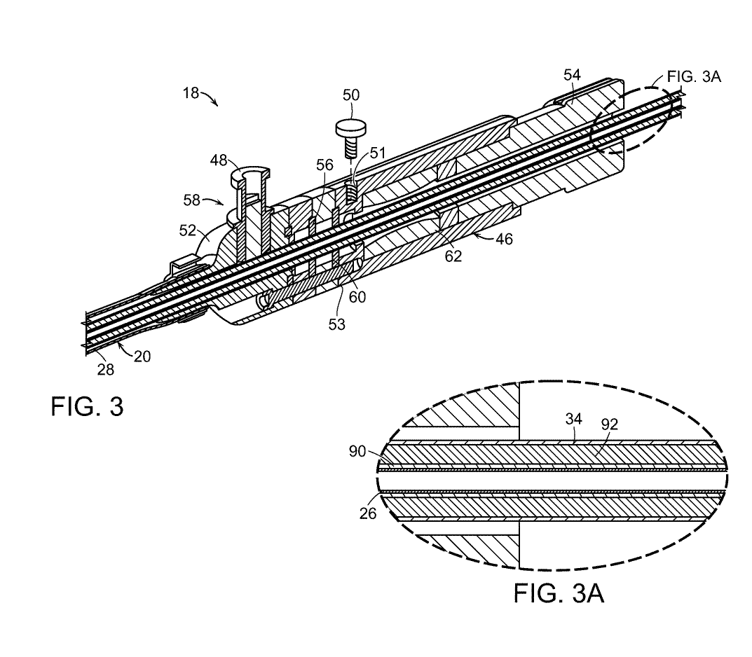

As shown in FIG. 3, hemostasis valve 18 of stent graft delivery system 10 includes hemostasis valve body 46 defining a central orifice through which distal sheath 34 and inner control tube 26 extend (FIG. 3A), and flush valve orifice 48 extending substantially normal to the central orifice, hemostasis valve body 46 is detachably fixable to handle 12 by suitable means, such as by, for example, release pin 50, which extends through internal lead screw assembly 40 into hemostasis valve 18 at opening 51, as shown in FIGS. 1 and 6. Returning to FIG. 3, cap 52 is coupled to a distal end 53 of the hemostasis valve body 46. Cap 52 defines a central orifice that is substantially aligned with the central orifice of hemostasis valve body 46 and through which distal sheath 34 and inner control tube 26 extend. Hemostasis valve knob 54 is threadably coupled to hemostasis valve body 46. Introducer sheath 20 extends distally from cap 52 and defines a lumen that is substantially aligned with the central opening of hemostasis valve body 46 and through which distal sheath 34 and inner control tube 26 extend. Wiper valve 56 is at the central opening of hemostasis valve 18 proximal to flush valve orifice 48. Wiper valve 56 forms a seal about inner control tube 26. X-valve 60 is at central opening of hemostasis valve 18 proximal to wiper valve 56. X-valve 60 forms a seal about a guidewire within inner control tube 26 upon withdrawal of the inner control tube 26 from hemostasis valve 18. Sheath valve 62 is at the central opening of hemostasis valve 18 and proximal to X-valve 60. Hemostasis sheath valve 18 is operable by activation of knob 54 to thereby seal the central opening by closing sheath valve 62. Wiper valve 56 operates to seal hemostasis valve 18 around distal sheath 34. Upon removal of distal sheath 34, X-valve 60 becomes the primary seal of hemostasis valve 18.

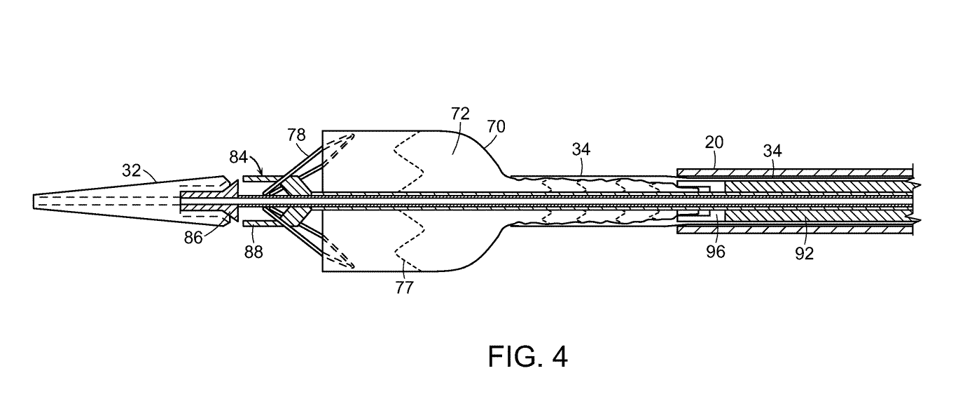

Referring back to FIG. 2, delivery system 10 further includes stent graft prosthesis 70 extending longitudinally within distal sheath 34, and radially about inner control tube 26 proximal to tip 32. Stent graft 70 includes luminal wall 72, proximal end 74, distal end 76, and a plurality of radial stents 77 extending longitudinally along luminal wall 72. (FIG. 2C) Retraction of distal sheath 34 within introducer sheath 20 exposes stent graft prosthesis 70, as can be seen in FIG. 4. In one embodiment, the plurality of stents 77 are self-expanding. In one embodiment, stents 77 include struts 79, wherein at least a portion of struts 79 define apices 81. (FIG. 2C) In one embodiment, self-expanding bare stent 78 extends proximally from proximal end 74 of stent graft wall 72. Bare stent 78 includes optionally, albeit not shown, a bare stent can extend distally from a distal end of wall 70. Typically, stents 77 and 78 are formed of a suitable material, such as nitinol, as is known to those skilled in the art.

Stent capture device 84 is at distal end 30 of inner control tube 26 and includes: distal capture portion 86 and proximal capture portion 88, wherein the proximal and distal capture portions define an opening that can releasably secure at least one apex 82 of bare stent 78, and apex release lumen 90 extending about inner control tube 26 and within distal sheath 34. Proximal capture portion 88 is fixed to apex release lumen 90 and extends through handle 12, whereby retraction of apex release lumen 90 will separate proximal capture portion 88 from distal capture portion 86, thereby releasing proximal end 74 of stent graft 70, such as by releasing at least one apex 82 of bare stent 78 of stent graft prosthesis 70. Delivery system 10 can further include intermediate tube 92 extending about apex release lumen 90 and within introducer sheath 20, wherein distal end 94 of intermediate tube 92 includes buttress 96 that abuts distal end 76 of stent graft prosthesis 70.

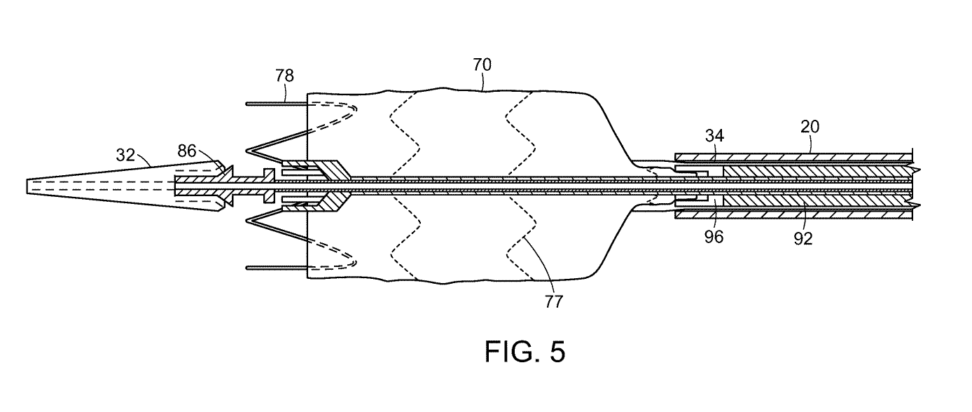

In another embodiment, the invention is a method for delivering a stent graft to a surgical site. The method includes, in one embodiment, the steps of directing stent graft prosthesis 70 within distal sheath 34 of a delivery system, such as delivery system 10 of FIGS. 1-10B, to an implantation site in an abdominal aortic aneurysm (AAA), a thoracic aortic aneurysm (TAA) or thoraco-abdominal aortic aneurysm (TAAA). Thereafter, distal sheath 34 is retracted from stent graft 70 by at least one of rotation of lead screw nut 44 in the direction 41 indicated in FIGS. 1 and Option I of FIG. 6, to result in retraction of distal sheath 34, as shown in FIGS. 2 and 4, thereby exposing stent graft 70 to the aneurysm. Alternatively, lead screw nut 44 can be slid directly back along handle 12, as shown in Option II of FIG. 6. In one particular embodiment, also shown in FIGS. 4 and 5, bare stent 78 of stent graft 70 is secured to distal end 30 of inner control tube 26 by stent capture device 84 and, in this embodiment, the method further includes the step of releasing stent graft 70 at an implantation site at, near, or adjacent to the aneurysm by moving stent capture device 84 from a closed position, shown in FIG. 4 to an open position, shown in FIG. 5. It is to be understood, however, that, although the example described herein and illustrated in the drawing employs a bare stent that is captured by stent capture device 84, other mechanisms can be employed in the alternative. For example, proximal end 74 of stent graft prosthesis 70 can be captured at proximal end 74 of luminal wall 72 by an interference fit within an orifice defined by tip 32 in an embodiment of stent graft 70 that includes no bare stent at proximal end 74. Alternatively, the stent graft can be captured by stent capture device 84, described above, where the stent graft includes a crown stent and clasping stent, as taught in U.S. Pat. No. 9,198,786, the relevant teachings of which are incorporated by reference in their entirety.

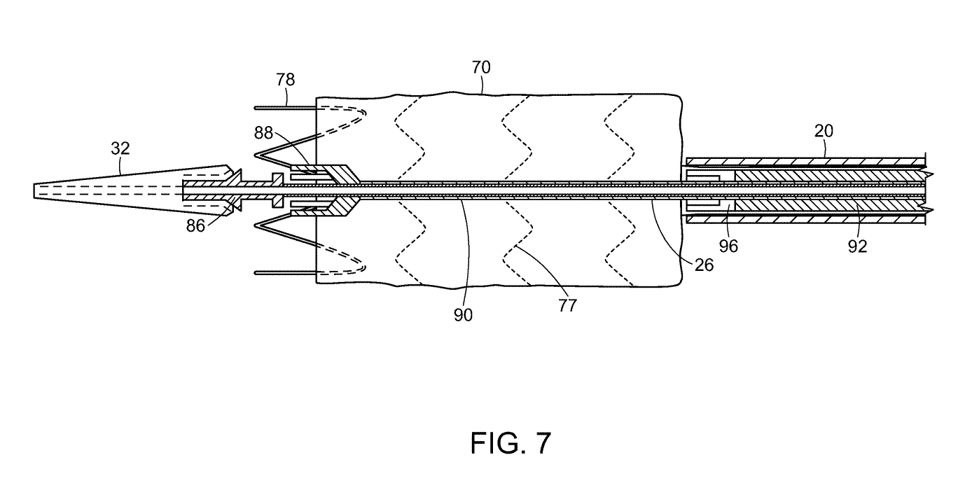

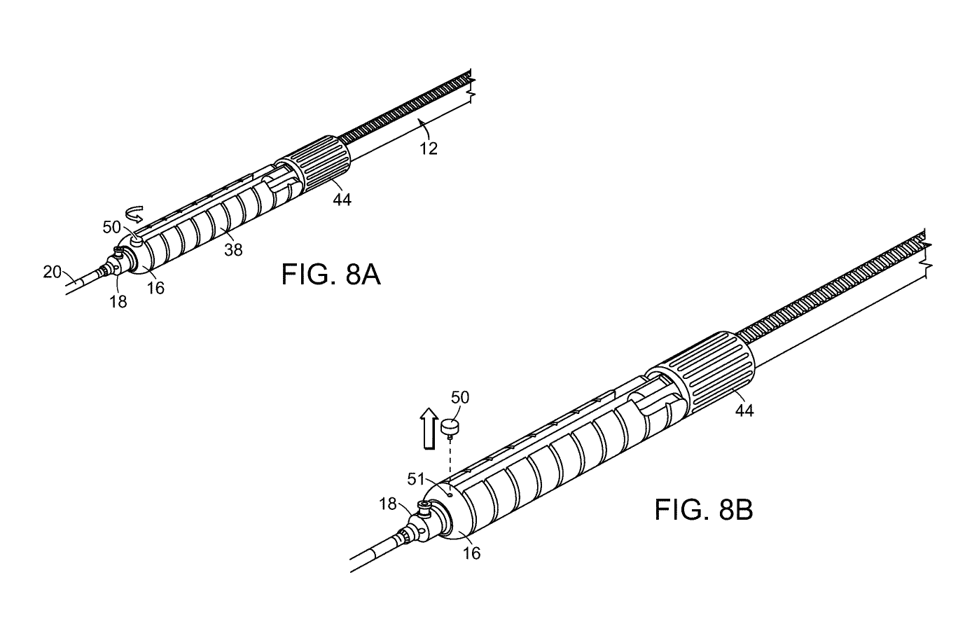

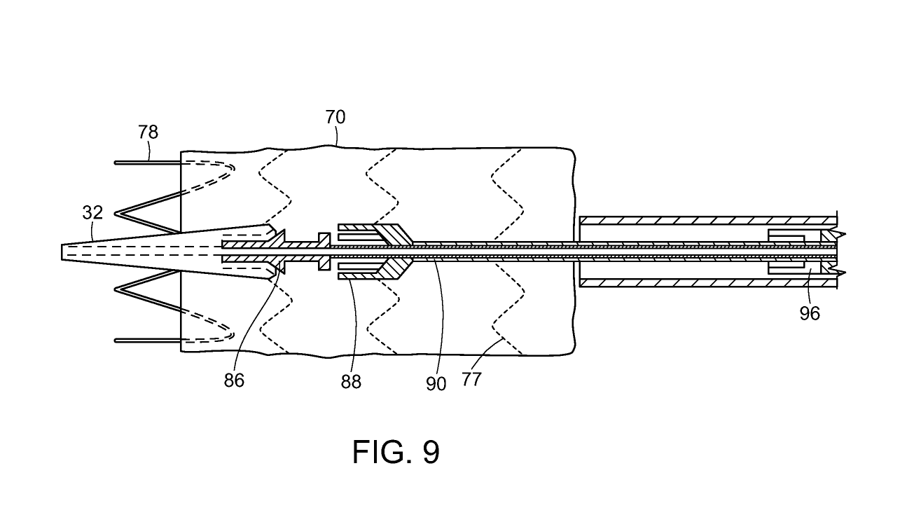

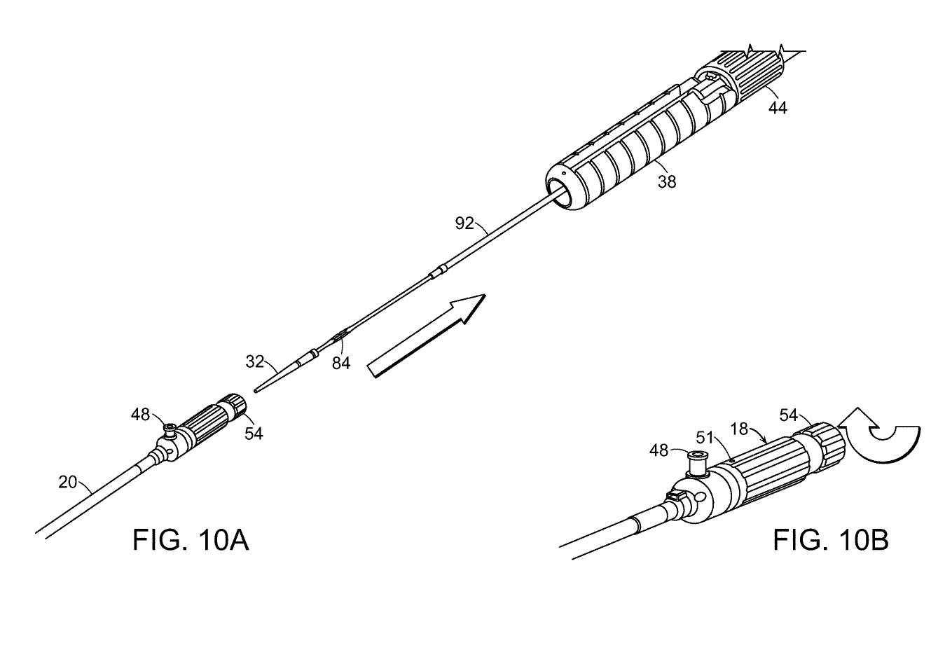

Thereafter, distal sheath 34 is fully retracted within introducer sheath 20 to fully release stent graft prosthesis 70, as shown in FIG. 7. Screw 98 is then removed, as shown in FIG. 8, to release hemostasis valve 18 from handle 12, whereby capture device 84, inner control tube 26, and tip 32 can be retracted through implanted stent graft 70, as shown in FIG. 9, and through introducer sheath 20 and hemostasis valve 18, as shown in FIG. 10. Hemostasis valve 18 is then closed by turning knob 54 (FIG. 3), as indicated by arrow 55 in FIGS. 1 and 10B, to prevent blood loss until other surgical steps, such as implantation of further stent graft components through hemostasis valve 20 and introducer sheath 20 are completed. Following implantation of any additional components of a stent graft prosthesis, such as legs of a bifurcated prosthesis (not shown), through hemostasis valve 18 and introducer sheath 20, hemostasis valve 20 and introducer sheath 20 can be removed from the subject to complete the surgical procedure.

The relevant teachings of all patents, published applications and references cited herein are incorporated by reference in their entirety. The relevant teachings of U.S. Pat. Nos. 8,292,943; 7,763,063; 8,308,790; 8,070,790; 8,740,963; 8,007,605; 9,320,631; 8,062,349; 9,198,786; 8,062,345; 9,561,124; 9,173,755; 8,449,595; 8,636,788; 9,333,104; 9,408,734; 9,408,735; 8,500,792; 9,220,617; 9,364,314; 9,101,506; 8,998,970; 9,554,929; 9,439,751 and U.S. patent application Ser. Nos. 14/226,005; 14/675,102; 15/099,974; 15/040,460; 14/575,673; 14/924,102; 15/166,818; 15/167,055; 14/736,978; 13/454,447; 15/384,663; 13/788,724; 15/417,467; 15/230,601; 14/272,818 and 14/861,479 are incorporated by reference in their entirety.

While this invention has been particularly shown and described with reference to example embodiments thereof, it will be understood by those skilled in the art that various changes in form and details made be made therein without departing from the scope of the invention encompassed by the appended claims.

What is claimed is:

1. A delivery assembly, comprising:
a) a handle defining a conduit;
b) an introducer sheath extending distally from the handle, the introducer sheath having a proximal end at the handle, and a distal end that is distal to the handle;
c) a substantially straight inner control tube extending through the conduit of the handle and from the distal end of the introducer sheath, the inner control tube having a proximal end that is proximal to the handle and a distal end that is distal to the distal end of the introducer sheath;
d) a tip fixed to the distal end of the inner control tube; and
e) a distal sheath that extends through the handle and the introducer sheath, and distally from the distal end of the introducer sheath and about the inner control tube to the tip, wherein the distal sheath has a diameter equal to or less than that of the introducer sheath, whereby retraction of the distal sheath through the introducer sheath and the handle exposes the inner control tube.

2. The delivery assembly of claim 1, wherein the handle includes a hemostasis valve component that seals the conduit.

3. The delivery system of claim 2, wherein the valve is a hemostasis valve.

4. The delivery assembly of claim 2, wherein the handle further defines a flushing port in fluid communication with the conduit and distal to the hemostasis valve.

5. The delivery assembly of claim 4, wherein the distal sheath is of a material that is more flexible than that of the introducer sheath.

6. The delivery assembly of claim 5, wherein the tip has a smaller outside diameter than an inside diameter of the introducer sheath and the conduit, whereby the distal sheath, the inner control tube and the tip can be retracted through the introducer sheath and the conduit, and thereby be removed from within the introducer sheath and the handle.

7. The delivery assembly of claim 1, further including a stent graft prosthesis extending longitudinally within and radially about the distal sheath, and about the inner control tube proximal to the tip, the stent graft including a luminal wall, a proximal end and a distal end, and a plurality of radial stents extending longitudinally along the luminal wall, whereby retraction of the distal sheath will expose the stent graft prosthesis.

8. The delivery assembly of claim 7, wherein the stent graft prosthesis includes a plurality of self-expanding stents.

9. The delivery assembly of claim 8, wherein the self-expanding stents are located at the proximal end and the distal end of the stent graft prosthesis.

10. The delivery system of claim 7, wherein the at least a portion of the stents include struts, and wherein at least a portion of the struts define apices.

11. The delivery system of claim 10, wherein a portion of the apices are exposed.

12. The delivery system of claim 11, further including a stent capture device at the tip, the capture device including:
a) a distal capture portion;
b) a proximal capture portion, wherein the distal capture portion and the proximal capture portion define an opening that can releasably secure the proximal end of the stent graft; and
c) an apex release lumen between the inner control tube and the distal sheath, the apex release lumen being fixed to the proximal capture portion and extending through the handle, whereby retraction of the release lumen will separate the proximal capture portion from the distal capture portion, thereby releasing the proximal end of the stent graft.

13. The delivery assembly of claim 12, wherein the capture device captures at least one apex of at least one stent of the stent graft, whereby retraction of the proximal capture portion from the distal capture portion releases the at least one apex and, consequently, the proximal end of the stent graft.

14. The delivery assembly of claim 13, further including an intermediate tube extending about the release lumen and having a distal end, the distal end including a buttress that is proximal to the capture device.

15. A method for delivering a stent graft to an implantation site, comprising the steps of:
a) directing a distal end of a delivery assembly to an implantation site in an abdominal aortic aneurysm, or a thoraco-abdominal aneurysm or a thoracic aortic aneurysm, the delivery assembly including,
i) a handle defining a conduit,
ii) an introducer sheath extending distally from the handle, the introducer sheath having a proximal end at the handle, and a distal end that is distal to the handle,
iii) a substantially straight inner control tube extending through the conduit of the handle and from the distal end of the introducer sheath, the inner control tube having a proximal end that is proximal to the handle and a distal end that is distal to the distal end of the introducer sheath,
iv) a tip fixed to the distal end of the inner control tube,
v) a distal sheath that extends through the handle and the introducer sheath, and distally from the distal end of the introducer sheath and about the inner control tube to the tip, wherein the distal sheath has a diameter about equal to or less than that of the introducer sheath, whereby retraction of the distal sheath through the introducer sheath and the handle exposes the inner control tube, and
vi) a stent graft that includes a luminal wall, a proximal and a distal end, and a plurality of radial self-expanding stents distributed longitudinally along the luminal wall, the stent graft extending about the inner control tube and within the distal sheath, wherein the proximal end of the stent graft is proximate to the distal end of the inner control tube; and
b) retracting the distal sheath from the stent graft, thereby delivering the stent graft to the implantation site in the aorta.

16. The method of claim 15, wherein the proximal end of the stent graft is secured to the distal end of the inner control tube by a capture device, and further including the step of releasing the stent graft at the implantation site by moving the capture device from a closed position to an open position.

17. The method of claim 16, wherein the capture device includes a distal capture portion at the distal end of the inner control tube, a proximal capture portion moveable along the inner control tube and which, in combination the distal capture portion, releasably captures the proximal end of the stent graft, and a release lumen that extends between the inner control tube and the distal sheath, whereby retraction of the release lumen separates the proximal capture portion from the distal capture portion to open and thereby release the stent graft at the implantation site, and further including the step of opening the capture device to thereby release the stent graft at the implantation site.

18. The method of claim 17, wherein the at least a portion of the stents of the stent graft includes stents define apices, at least a portion of which are exposed and captured by the capture device, and whereby the stent graft is released by release of the apices from the capture device.
